(19) United States
(12) Patent Application Publication
(10) Pub. No.: US 2005/0208147 A1
(43) Pub. Date: Sep. 22, 2005

(54) Isolation of bivittoside D from sea cucumber and activity thereof

(75) Inventors: Lakshmi Vijay, Lucknow (IN); Ajet Saxena, Lucknow (IN); Kartikay Pandey, Lucknow (IN); Padmanabhan Madhusudanan Kunnath, Lucknow (IN); Mahendra Nath Srivastava, Lucknow (IN); Zafar Kamal Khan, Lucknow (IN); Pooja Jain, Lucknow (IN); Gopal Gupta, Lucknow (IN); Janaki Dulari Dhar, Lucknow (IN)

Correspondence Address: William R. Evans;Ladas & Parry, 26 West 61 Street, New York, NY, 10023, US

(73) Assignee: Council of Scientific and Industrial Research

(21) Appl. No.: 10/806,078

(22) Filed: Mar. 22, 2004

(51) Int. Cl. A61K 35/12; A61K 31/704

(52) U.S. Cl. 424/520; 514/33

(57) ABSTRACT

The present invention provides a process for the isolation of Bivittoside-D, a triterpenoid saponin from the sea cucumber Bohadschia vitiensis and its potent spermicidal and fungicidal activity.

### FIELD OF THE INVENTION

The present invention provides a process for the isolation of Bivittoside-D, a triterpenoid saponin from the sea cucumber Bohadschia vitiensis and its potent spermicidal and fungicidal activity. The present invention also relates to a fungicidal composition comprising bivittoside D isolated from sea cucumber. The present invention also relates to a spermicidal composition comprising a spermicidal amount of a triterpenoid saponin Bivittoside D isolated from sea cucumber.

### BACKGROUND OF THE INVENTION

Sea cucumbers are utilized as therapeutic agents in the Malaysian peninsula and as a food, especially among the ethnic people of Sabah. Literature review revealed that two different research groups have worked on the chemistry of the Bohadschia vitiensis (semper).

In one report, Cuvier's organs of Bohadschia vitiensis were extracted with ethanol and the crude extracts on hydrolysis yielded four genins. Three of these genins are known from other species and were identified as (seychellogenin, 15-oxido-holothurinogenin and 24, 25-dehydro holothurinogenin) and the fourth one was found a new sapogenin (Clastres, A. A. Poupt, c. Poltier, P. et al. Expertentia, (1978) 34 (18) 973-4(7r). However, there was no report on the bioactivities of these genins, fractions etc. From another species of B. argus (jaegar), bivittoside has been isolated in 21% yield based on methanolic extract of the body wall whereas Bivittoside D was 37% in yield based on cuvierian tubules of the sea cucumber. It is worth mentioning that cuvierian tubules of sea cucumber contain most of the saponin and constitute 50% saponin part of whole sea cucumber. [Kitagawa, I., Kobayashi, M., Hori, M. and Kyogoku, Y. Chem..Pharm..Bull. 37, 61(1989)]. Neither the details of its isolation method nor its bioactivity have been reported. Another report describes the structures of four new triterpenoid oligosaccharides, bivittoside a, b, c, and d from the sea cucumber B. bivittata (mitsukuri) (Kitagawa, J., Kobayashi, M. Kyogoku, Y., Chem. Pharm. Bull. 29, 282-85 (1981). The methanolic extract of the cuverian tubles of B. bivittata afforded bivittoside a, b, c, and d after solvent fractionation and on chromatographic separation, in 2, 2, 2 and 8% yields, respectively. Their structures have been determined by Spectrochemical evidences. (Kobayashi, M., Hori, M., Kan, K., Yasuzawa, T., Matsui, M., Suzuki, S, Fkitagawa, I. Chem. Pharm. Bull. 1991, 39, 2282 -87.)

In another report Bivittoside c and d are reported from B. argus, B. marmorata, B. vitiensis and B. tenuissina collected from Indian Ocean and characterized by acid hydrolysis and Spectral analysis. (Antonov, A. S, Stonik, V. A. Khim Prir Soedin 1986, 379-80). However, none of the publications report the isolation of bivittoside without chromatography and are mainly of academic interest. The prior art reports do not attempt isolation of bivittoside d on pilot scale for drug development.

Among the four bivittosides (a,b,c, & d) only d was found to exhibit significant antifungal activities. The fungus tested were Aspergillus niger, A. oryzae, Penicillium citrinum, P. chrysogenum, Mucor spinescens, Cladosporium herbarium, Rhodotorula rubra, Trichophyton mentagrophytes, T. rubrum, Candida albicans and C. utilis [Kitagawa, I., Kobayashi, M, Hori, M and Kyogoku, Y. Chem Pharm. Bull. (1989), 37, 61-67.]

This saponin may be employed as local antifertility agent and a single sea cucumber drug in the form of contraceptive cream, jelly or water-soluble film to prevent pregnancy in women and as a fungicide cream. The use of contraceptive creams, jellies or water-soluble film containing a spermicide provides an easy and convenient method of preventing pregnancy in women. Most of the currently used spermicidal agents are either costly or cause undesirable side effects e.g. irritation, eczema, dermatitis or skin rash. Besides, the fungicidal activity of the saponin especially that against Candida albicans can offer prophylactic contraception as C. albicans causes a very common vaginal infection in the human population. The saponin can also be utilized as a fungicide cream or jelly.

### OBJECTS OF THE INVENTION

The main object of present invention is to obtain non-toxic potent spermicides and fungicides from natural sources.

It is another object of the invention to provide an improved process for the isolation of Bivittoside D a triterpenoid saponin from the sea cucumber Bohadschia vitiensis.

### SUMMARY OF THE INVENTION

Accordingly, the present invention provides a process for the isolation of Bivittoside D from Bohadschia vitiensis, said process comprising soaking B. vitiensis material in a first polar solvent, filtering the material and decanting the solvent followed by soaking the material in a second aqueous polar solvent, extracting the material by filtration, concentrating the extracted material under reduced pressure to obtain a thick viscous crude extract, euting the crude extract followed by crystallization to obtain a pure saponin Bivittoside D.

In one embodiment of the invention, the polar organic solvent used for extraction is selected from the group consisting of methanol, ethanol, propanol, butanol, water and any mixture thereof.

In another embodiment of the invention, the first polar solvent comprises of n-butanol saturated with water.

In yet another embodiment of the invention, the viscous crude extract is eluted using silica gel or gel filtration column and wherein the eluent used is selected from the group consisting of chloroform, methanol, ethanol, water and any mixture thereof.

In another embodiment of the invention, the yield of the bivittoside D is in the range of 25-30%.

The present invention also relates to a spermicidal composition comprising an effective amount of bivittoside D and one or more pharmaceutically acceptable additives.

In one embodiment of the invention, the bivittoside D is used in an amount of 0.01 % to 1.0% aqueous solution.

The invention also relates to the use of bivittoside D isolated from Bohadschia vitiensis as a spermicidal agent.

In one embodiment of the invention, the bivittoside D is used as such as an spermicidal agent.

In one embodiment of the invention, the bivittoside D is used in an amount of 0.01% to 1.0% aqueous solution.

In another embodiment of the invention, the bivittoside based composition is in the form of a cream, jelly, free-flowing powder, solution, suspension, and alcoholic extract.

The present invention also provides a fungicidal composition comprising a active amount of bivittoside D isolated from Bohadschia vitiensis and one or more conventional additives.

In one embodiment of the invention, the amount of bivittoside D present in the composition is in the range of 0.39 mg/ml to 12.57 mg/ml.

The present invention also relates to the use of bivittoside D isolated from Bohadschia vitiensis as a fungicidal compound against Candida albicans, Cryptococcus neoformans, Sporothrix schenckii, Aspergillus fumigatus and Frichophyton mentagrophytes pathogens.

In another embodiment of the invention, the bivittoside based composition is in the form of a cream, jelly, free-flowing powder, solution, suspension, and alcoholic extract.

### DETAILED DESCRIPTION OF THE INVENTION

The present invention consists in obtaining spermicidal and fungicidal pure saponin in a stable and free flowing non-hygroscopic solid form from the sea-cucumber Bohadschia vitiensis of the Indian Ocean coast.

The present invention is based on the observation that methanolic extract of sea cucumber, which contains saponin of lanostane type, instantaneously kill the human sperm on coming in contact to the latter. The saponin bivittoside-d which is the active constituent of this sea cucumber can be incorporated in spermicides either as a saponin or together with a water-soluble or water-dispersible base in the form of a vaginal cream or jelly or as a thin film which in presence of moisture dissolves or swells up to slimy gel like mass.

The saponin isolated from sea cucumber (bivittoside-d) can also be used as a fungicide cream.

In search towards new biologically active substances from marine sources, attention has been paid to echinoderms, and amongst them, to sea cucumbers (class—Holothuridae). These invertebrates have shown to contain a variety of triterpene glycosides of lanostane class with a distinctive g-lactone skeleton named as holostane and sugar chain composed of up to six monosaccharide units, principally D-xylose, D-glucose, D-quinovose, D-3-0-methylglucose and D-3-0-methyl xylose. In addition sulphate groups can also be found at certain positions of the aglycone and/or sugar moiety.

Bohadschia vitiensis (semper) belongs to Phyllum echinodermata, class holothuroidea, order aspidochirotida, family holothuridae, genus Bohadschia and species vitiensis.

The sea cucumber was collected from Corbyn's cave in South Andaman coast under 2-3 meter depths in the month of June. Specimen sample has been preserved in the herbarium of Botany division, Central Drug Research Institute, Lucknow with the Field No. 21455, Botany Serial No. 326 and CDRI Code No. CDR-258.

The present invention provides a process for the isolation of bivittoside d, a triterpenoid saponin from the sea cucumber Bohadschia-vitiensis (semper) and its potent spermicidal and antifungal activity.

Freshly collected organisms (whole body 2.0 kg) were washed with water, soaked in aqueous methanol and were brought to the laboratory, where the methanol was decanted. The organisms were cut into small pieces, filled in glass percolators and were soaked in fresh methanol. After repeating 3-4 times, all the combined extracts were evaporated. The residue thus obtained was coded as A001 (60 g). The pieces were further percolated with chloroform-methanol (1: 1, v/v) 5 times and combined extracts were concentrated under reduced pressure (10-50 mm Hg) below 500° C. to a greenish viscous mass, which was finally dried under high vacuum and coded as A002 (30 g).

General Method:

The crude extract A001 (25 g) was fractionated by macerating with hexane (5×25 ml), chloroform (5×25 ml), n-butanol (5×25 ml) successively and fractions obtained thereof were evaporated to dryness and designated as F003 (0.25 g), F004 (1.25 g), F005 (2.25 g) and the residual mass F006 (20 g) respectively. All these fractions were tested for spermicidal and antifungal activity in in-vitro models. F005 showed the promising spermicidal activity. For bioactivity fractions F005 and F006 were combined and chromatographed over a column of silica gel and bivittoside-d (5.0 g) was obtained in pure form from methanol-water (9:1) eluent. Bivittoside-d was named as K007 (25% yield based on methanolic extract obtained from the whole body of sea cucumber). It showed promising spermicidal (Table 1) and antifungal activity (Table 2) in in vitro models.

Bivittoside-D is a lanostane triterpenoid having 6 monosaccharide units. The structure of the compound was established on the basis of Physio-chemical data, acid hydrolysis of saponin, identification of sugar units and aglycon, m.p.. The structure of bivittoside d is as follows: 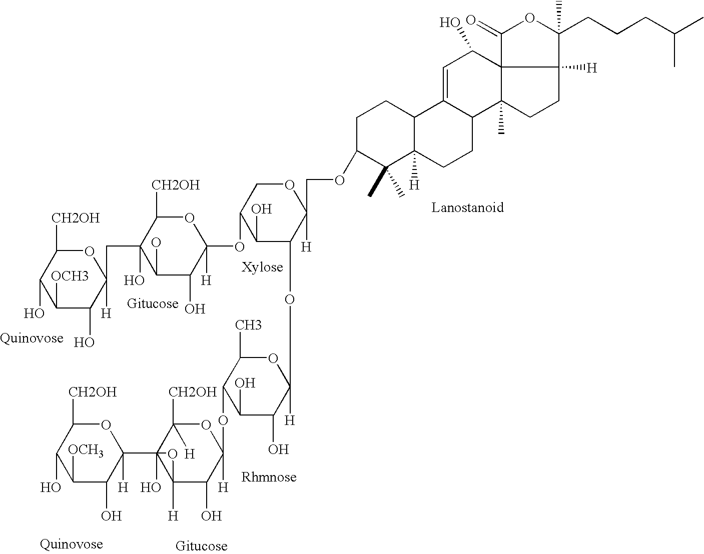
Spermicidal Activity Evaluation: In Vitro

The spermicidal activity was determined by the Sander Cramer assay (Sander, F. V., Cramer, S. D., Human Fertility, (1941) Vol. 6, p-134) using physiological saline (0.85% NaCl). Aqueous solutions (1.0% i.e. 10 mg/ml) of different fractions of B. vitiensis were prepared in physiological saline, which were diluted further up to a concentration of 0.01%. Human semen (0.2 ml) was mixed with each test solution (1.0 ml) separately and vortexed for 10 s. A wet mount of each preparation was immediately examined under a phase contrast microscope. The lowest spermicidal concentration that completely immobilized 100% of the spermatozoa within 20 s. was referred to as MEC (minimum effective concentration). The activity was confirmed in three different semen samples with >50×10⁶/ml sperm count and >50% motility.

The crude extract (A001) showed 100% mortality of human sperms at 0.01% concentration, A002 was less effective showing 100% efficacy at 0.1% concentration. Among the four fractions prepared from A001 i.e. F003, F004, F005 and F006, the butanol soluble (F005) as well as insoluble (F006) fractions showed mec 0.05%. The mec of pure compound bivittoside d was obtained 0.05%.

TABLE 1
Spermicidal activity in extracts/fractions/pure
compounds of Bohadschia vitiensis

| | | Mec |
|---|---|---|
| S. No. | Name of the fraction | (%) |
| 1. | A001 (Aqueous Methanolic extract.) | 0.01 |
| 2. | A002 (Methanol-Chloroform (1:1) extract.) | 0.1 |
| 3. | F003 (Successive Hexane soluble fraction from A001 | 1.0 |
| 4. | F004 (Chloroform soluble Fraction from A001) | 1.0 |
| 5. | F005 (n-Butanol soluble fractions From A001) | 0.05 |
| 6 | F006 (n-Butanol insoluble fractions from A001) | 0.05 |
| 7. | K007 (Pure compd. Bivittoside-d) | 0.05 |

In another method F005 and F006 were taken in methanol and decolorized with animal charcoal (Ranbaxy, India) and the filtrate was concentrated and saponin fractions were precipitated was done by addition of acetone.

Antifungal Activity Evaluation: In Vitro

Extracts/fractions/pure compounds obtained from B. vitiensis were tested in in-vitro antifungal activity against five pathogenic fungi in terms of minimum inhibitory concentration (mic,mg/ml) following reference methods. Mics for yeasts: Candida albicans (ca), Cryptococcus neoformans (cn) and Sporothrix schenckii (spo) were determined by broth micro-dilution method according to guide lines of nccls documents m-27a1 (National Committee for Clinical Standards (1997) reference method for broth dilution. Antifungal susceptibility testing of yeast approved standards document m-27a, nccls, wayne, Pa.) In RPMI-1640 medium buffered at pH 7.0 with 0.165 m morpholine propane sulphonic acid (mops: Sigma USA moris; Mo.). Assays were performed in flat bottomed 96 wells microlitre plate. Stock solution of products were prepared in DMSO and diluted by two-fold serial dilution. The working suspension (0.5×103 to 2.5×103) of the innoculum, prepared spectrophotometrically (0.5 mc Farland turbidity standard) was made by a 1:50 dilution followed by a 1:20 dilution of the seeded broth in RPMI-1640. Suitable controls were maintained simultaneously. Plates were incubated at 35° C. and observed visually at 24 hr for ca and 48 hr cn and spo. The susceptibilities of molds Aspergillus fumigatus (af) and Frichophyton mentagrophytes (tm) were determined by the NCCLS proposed document m-38p2 (National Committee for Clinical laboratory Standards (1997) reference method for broth dilution antifungal susceptibility testing of Aspergillus, proposed standards document m-38 p, nccls, wayne, Pa.). In brief, cultures were grown on sabouraud dextrose agar at 35° C. until sporulation occurred. Spores were suspended in 0.85% normal saline and diluted in RPMI-1640 to yield 104 cf p/ml. The mics for molds were defined as the lowest concentration that significantly reduced growth compared to the drug-free control.

The crude extracts A001 and A002 showed broad spectrum activity against pathogenic yeast and mycelial fungi (Table-2). The extract A001 exhibited the highest activity against cn and af (mic 7.8 mg/ml). On fractionation, the activity was found to be increased in tm and spo particularly with the fraction F006 (mic 0.39mg/ml). The pure compound bivittoside d, K007 was found to be active against all the tested fungi and the best activity was observed in case of tm (mic 1.56 mg/ml) followed by ca (mic 3.12 mg/ml) cn and spo (mic 6.25 mg/ml) and af (mic 12.57 mg/ml) (Table-2). Overall, the pure compound showed promising activity in-vitro models of the said fungal pathogens.

TABLE 2
In vitro minimum inhibitory concentration in extracts/fractions/pure
compound (mic mg/ml) of B. vitiensis

| | Mics (μg/mi.) against pathogenic fungi | | | | |
|---|---|---|---|---|---|
| Sample | ca | Cn | spo | tm | Af |
| A001* | 125 | 7.8 | 31.2 | 31.2 | 7.8 |
| A002* | 3.12 | 6.25 | 12.5 | 6.25 | 12.5 |
| F003* | 500 | 500 | 500 | 62.5 | 500 |
| F004* | 500 | 500 | 500 | 15.6 | 500 |
| F005* | 500 | 62.5 | 6.25 | 15.6 | 500 |
| F006* | 50 | 0.39 | 0.39 | 0.39 | 3.12 |
| K007* | 3.12 | 6.25 | 1.56 | 1.56 | 12.5 |

Ca = candida albicans,
cn = cryptococcus neoformans,
Spo = sporothrix schenckii,
tm = trichophyton mentagrophytes,
Af = aspergillus fumigates.
*tested at 1000-1.95 mg/ml concentration.
** tested at 100-0.19 mg/ml concentration.

A process for the isolation of a saponin useful as a spermicide drug from the whole body of Bohadschia vitiensis which comprises, soaking of the freshly collected organism Bohadschia

This saponin has been isolated for the first time from Bohadschia-vitiensis in good yield (25 to 30%). In such a high yield it can be developed as economically viable drug.

The process for the isolation of the saponin bivittoside-d from the sea cucumber Bohadschia vitiensis involves the following steps—
1. Washing of sea cucumber with tap water for removal of mainly common salts and other inorganic sea salts.
2. Transporting it to the place of work in methanol at room temp. (35-42° C.) in a suitable sealed containers.
3. Chopping of the material into small pieces.
4. Soaking it into methanol or other polar organic solvents three to four times at room temperature.
5. Soaking it into mixture of polar organic solvents and water.
6. Decantation, filtration of the extract (step 5, 6) and removal of solvent till dryness under vacuo (10-50 mm hg) by conventional methods as to get extracted residue.
7. Dividing the extracted residue into non-polar, semi-polar and polar extracts by successive maceration with respective solvents.
8. Purification of the polar compound (saponin) from the polar fraction by precipitation (using combination of solvents), column chromatography.
9. Removal of inorganic salts by gel filtration /organic solvents.

### EXAMPLE 1

Small pieces of Bohadschia-vitiensis (100 g) were soaked in methanol (200 ml×5) overnight and all the extracts were mixed, decanted, filtered and evaporated to dryness (wt. 30 g).

It was dissolved in methanol again (100×5 times) to filter out the methanol insoluble inorganic salts. The methanolic extract was combined and methanol was removed under vacuo. The light brown residue (wt. 2.5 g) thus obtained was successively macerated with chloroform and n-butanol. Chloroform fraction was rejected and n-butanol fraction after removal/recovering of most of the butanol was dissolved into 100 ml methanol. To it 30 ml acetone was added gradually with stirring. The light brown precipitate that separated out was filtered (wt. 600 mg). This powder was again dissolved into methanol (20 ml) and (30 ml) acetone was added to get colorless or pale yellow powder. On SiO₂ TLC it showed single spot using the solvent system chloroform: methanol (35: 10:2,v/v). It was coded as K007.

It was crystallized (methanol-chloroform 3-H₂O, 8:2:1) as colorless solid mp. 220-2210c. HPLC analysis indicated it to be a single compound.

The overall yield of bivittoside d was 30% based on n-butanol extract of the organism.

### EXAMPLE 2

Methanolic extract was obtained from 100 g of the marine organism Bohadschia-vitiensis as per example 1. The methanolic extract (3.0 g) still consisted of some inorganic salts. This extract in methanol was loaded on to Sephadex lh-20 column packed in methanol-H₂O (9:1) and eluted with methanol-H₂O (9:1). In all twenty fractions, 25 ml each were collected and monitored with SiO₂-TLC plates using chloroform-methanol-water (35:10:2) solvent system. First and last six fractions mainly contained inorganic salts/undesirable compounds and were therefore rejected. Fraction 10-15 contained saponin bivittoside-d. These fractions were combined and solvent was removed to get a colorless residue in the yield of 500 mg. It was crystallized as colorless solid, mp.220-2210c, over all yield was calculated to be around 25%.

### EXAMPLE 3

Ethanol (95% alcohol) extract was obtained from 100 g Bohadschia vitiensis as per example 1. The concentrated ethanolic extract was dissolved in 500 ml of ethanol-water (1:1). The insoluble residue was rejected and the ethanol/water soluble portion was partitioned with chloroform (500 ml×3) followed by extraction with n-butanol (500 ml×5). n-butanol extract and ethanol: water extract both were concentrated to dryness to get light brown solid mass. The ethanol-water concentrated powder still contained bivittoside-d and therefore continuously extracted with butanol in a solid-liquid extractor (Soxhlet extractor) using n-butanol as solvent. The total combined concentrated butanol extract was dissolved in ethanol (25 ml) and to it ether was gradually added to precipitate the saponin. The precipitate was centrifuged and crystallized as pale yellow solid, m. P. 220-22° C. Overall yield was 25%.

Advantages:

The main advantages of the present invention are—
(1) Bohadschia vitiensis is a new, high yielding source of bivittoside d and has been isolated for the first time.
(2) Saponin bivittoside d present in Bohadschia vitiensis is in high yield (25-30% based on the weight of the whole organism) which has an advantage.
(3) Marine organism is found only at a depth of 2-3 meters. Therefore collection of the material is very easy.
(4) Saponin is quite stable for long time, therefore, it can be used as a cream for the use as a spermicide and fungicide.
(5) It is a free flowing powder.

1. A process for the isolation of Bivittoside D from Bohadschia vitiensis, said process comprising soaking B. vitiensis material in a first polar solvent, filtering the material and decanting the solvent followed by soaking the material in a second aqueous polar solvent, extracting the material by filtration, concentrating the extracted material under reduced pressure to obtain a thick viscous crude extract, euting the crude extract followed by crystallization to obtain a pure saponin Bivittoside D.

2. A process as claimed in claim 1 wherein the first polar organic solvent used for extraction is selected from the group consisting of methanol, ethanol, propanol, butanol, water and any mixture thereof.

3. A process as claimed in claim 1 wherein the first polar organic solvent used for extraction is selected from the group consisting of methanol, ethanol, propanol, butanol, water and any mixture thereof.

4. A process as claimed in claim 1 wherein the first polar solvent comprises of n-butanol saturated with water.

5. A process as claimed in claim 1 wherein the viscous crude extract is eluted using silica gel or gel filtration column and wherein the eluent used is selected from the group consisting of chloroform, methanol, ethanol, water and any mixture thereof.

6. A process as claimed in claim 1 wherein the yield of the bivittoside D is in the range of 25-30%.

7. A spermicidal composition comprising an effective amount of bivittoside D and one or more pharmaceutically acceptable additives.

8. A composition as claimed in claim 7 wherein the bivittoside D is used in an amount of 0.01% to 1.0% aqueous solution.

9. Use of bivittoside D isolated from Bohadschia vitiensis as a spermicidal agent.

10. Use as claimed in claim 9 wherein the bivittoside D is used as a spermicidal agent in pure form.

11. Use as claimed in claim 9 wherein the bivittoside D is used in an amount of 0.01% to 1.0% aqueous solution.

12. A composition as claimed in claim 7 wherein the composition is in the form of a cream, jelly, free-flowing powder, solution, suspension, and alcoholic extract.

13. A fungicidal composition comprising a active amount of bivittoside D isolated from Bohadschia vitiensis and one or more conventional additives.

14. A composition as claimed in claim 13 wherein the amount of bivittoside D present in the composition is in the range of 0.39 mg/ml to 12.57 mg/ml.

15. Use of bivittoside D isolated from Bohadschia vitiensis as a fungicidal compound against Candida albicans, Cryptococcus neoformans, Sporothrix schenckii, Aspergillus fumigatus and Frichophyton mentagrophytes pathogens.

16. A composition as claimed in claim 13 wherein the composition is in the form of a cream, jelly, free-flowing powder, solution, suspension, and alcoholic extract.
